(19) United States
(12) Patent Application Publication
(10) Pub. No.: US 2015/0230716 A1
(43) Pub. Date: Aug. 20, 2015

(54) DEVICE AND METHOD FOR MONITORING A PATIENT'S VASCULAR ACCESS, HAVING A WOVEN MOISTURE SENSOR WITH A MONITORING SECTION

(71) Applicant: Fresenius Medical Care Deutschland GmbH, Bad Homburg (DE)

(72) Inventor: John Heppe, St. Wendel (DE)

(21) Appl. No.: 14/391,218

(22) PCT Filed: Apr. 11, 2013
(86) PCT No.: PCT/EP2013/001058

§ 371 Date: Jan. 2, 2015

Related U.S. Application Data
(60) Provisional application No. 61/622,677, filed on Apr. 11, 2012.

(30) Foreign Application Priority Data
Apr. 11, 2012 (DE) 10 2012 007 082.9

(51) Int. Cl. A61B 5/02; A61M 1/36; A61B 5/00

(52) U.S. Cl. CPC A61B 5/02042; A61B 5/6801; A61M 1/3656

(57) ABSTRACT

The present invention proposes another woven moisture sensor having at least one conductor for monitoring a patient's vascular access for blood loss to the surroundings, in particular for use in an extracorporeal blood treatment.

The inventive moisture sensor has a monitoring section of the conductor which is produced by weaving, which is a high-resistance terminating resistor in a first embodiment and in a second embodiment is a tongue of the moisture sensor, which is provided and configured for cutting out the moisture sensor.

### FIELD OF THE INVENTION

The invention relates to the field of monitoring a patient's vascular access for blood loss into the surroundings by means of a woven moisture sensor, in particular in extracorporeal blood treatment, and an evaluation device for detecting moisture at the patient's vascular access.

### STATE OF THE ART

Various types of blood treatment devices are known. The known blood treatment devices include, for example, devices for hemodialysis, hemofiltration and hemodiafiltration. During an extracorporeal blood treatment, the blood flows from an arterial patient's vascular access through an arterial cannula and through an arterial line of the extracorporeal blood circulation into a blood treatment unit, for example, a dialyzer, and after flowing through the blood treatment unit, it flows back through a venous line of the extracorporeal blood circulation and through a venous cannula into the patient's venous vascular access. Monitoring of patients during an extracorporeal blood treatment for blood loss to the surroundings is a constant challenge for the staff at a dialysis clinic. One particular fear is accidental leakage of blood into the surroundings when the returning venous cannula is disconnected or dislocated from the venous vascular access. Various methods and devices for monitoring for blood loss to the surroundings from a patient's vascular access during a blood treatment session are therefore known from the state of the art. For example, there are known moisture sensors which can be glued to the puncture site of the vascular access. These known moisture sensors are based on the principle that the electrically conductive blood reduces the electrical resistance between two electrodes in the moisture sensors and the electrical resistance is monitored by an evaluation unit.

The document WO 2010/091852 A1 by the applicant Fresenius Medical Care Deutschland GmbH describes a moisture sensor for monitoring a patient's vascular access, in which the printed conductors and a high-resistance terminating resistor are applied subsequently by printing onto a nonwoven, for example, as a conductive printing ink or print paste. The printed terminating resistor serves to test the function of the printed conductors when the moisture sensor is dry. One disadvantage of the printed terminating resistor is the high effort for printing the terminating resistor in an accurate position combined with high production costs. Another disadvantage is the risk of local breaks or micro-breaks in the printing paste under mechanical loading of the moisture sensor, which can cause a significant increase in the electrical resistance during the use of the sensor.

The document WO 2011/116943 A1 of the applicant Fresenius Medical Care Deutschland GmbH describes a moisture sensor manufactured by weaving for monitoring a patient's vascular access. With this moisture sensor the conductors are implemented by electrically conductive warp and weft threads in a partially multilayer woven fabric. Electrically conductive threads usually have the purpose of conducting the electrical current in woven fabrics with the lowest possible electrical resistance. To this end the known fibers contain silver for example. Such a moisture sensor is provided for use with an external terminating resistor integrated into a terminal, namely an SMD (surface-mounted device) for testing the function of the conductors in the dry state of the moisture sensor. The moisture sensor produced by weaving must rely on the external terminating resistor because the integration of a defined terminating resistor into such a moisture sensor with the required high reproducibility, e.g., that of an SMD, would be complex and would to some extent defeat the substantial cost advantages of the production process by weaving. Such a moisture sensor would have four terminal contacts such that two terminal contacts must be provided for contacting the SMD in the terminal. The International Patent Application PCT/EP2011/003044 by the applicant Fresenius Medical Care Deutschland GmbH describes such a terminal for a moisture sensor for monitoring a patient's vascular access with two terminal contacts for the conductors and two additional terminal contacts for the terminating resistor integrated into the terminal. The disadvantage of these terminals is the increased cost for the two terminal contacts of the integrated terminating resistor. Another terminal for a moisture sensor to be placed on a patient's skin for monitoring a patient's vascular access is described in the German patent application of the applicant Fresenius Medical Care Deutschland GmbH which was still unpublished as of the filing date of the present patent application and which bears the application number DE 10 2011 113839.4.

The disadvantage of a terminal cable or a terminal having an integrated terminating resistor for a moisture sensor without an integrated terminating resistor is the increased manufacturing effort combined with increased manufacturing costs.

A weaving method for producing a plurality of moisture sensors for monitoring a patient's access is described in the German patent application of the applicant Fresenius Medical Care Deutschland GmbH which bears the application number DE 10 2011 113838.6 and was still unpublished as of the filing date of the present patent application.

Reference is made to the full content of the documents
WO 2010/091852 A1,
WO 2011/116943 A1,
   and the patent applications
DE 10 2011 113838.6 and
DE 10 2011 113839.4 and
PCT/EP2011/003044
   in the present patent application.

### OBJECTS OF THE PRESENT INVENTION

A generic woven moisture sensor has at least one conductor produced by weaving in a multilayer woven fabric such that the at least one conductor is filmed from conductive warp and conductive weft fibers in the woven fabric which otherwise consists of electrically nonconductive fibers, and the conductive warp fibers and the conductive weft fibers are woven together in an electrically conductive pattern at selected contact points. One object of the present invention is to advantageously improve upon a generic woven moisture sensor and overcome disadvantages of the known woven moisture sensors from the state of the art.

Another object of the present invention is to provide a generic moisture sensor produced by weaving in which the function of the conductors can be tested in the dry state without necessitating an external terminating resistor.

Another function of the present invention is to provide a generic moisture sensor which is produced by weaving with an integrated terminating resistor, so that the manufacturing complexity is low.

Another object of the present invention is to provide a generic woven moisture sensor with which a defined terminating resistor is integrated into the woven fabric and the ohmic resistance of the terminating resistor is reliably reproducible within given tolerances.

Another object of the present invention is to reduce the manufacturing costs for an evaluation device for monitoring a patient's vascular access with a generic woven moisture sensor.

Another object of the present invention is to provide a generic woven moisture sensor which is robust with respect to mechanical loads.

### THE PRESENT INVENTION

The solution to these problems is achieved according to the invention with the features of the Independent Patent Claims 1, 10, 13 and 14. Advantageous embodiments are subjects of the dependent claims. The advantages of the inventive moisture sensor according to Claim 1 can be achieved without being diminished with the evaluation unit according to Claim 10 and the device for extracorporeal blood treatment according to Claim 13 as well as the method according to Claim 14.

So far there has hardly been any experience with high-resistance fibers in electrically conductive woven fabrics in the state of the art. However, investigations by the applicant have shown that a sufficient reproducibility of a high-resistance terminating resistor produced only by weaving can be successful if, firstly, the thread of the terminating resistor is upgraded with a high-resistance polymer coating, and secondly, the effective length of the high-resistance terminating resistor in the fabric is defined and limited precisely through local conductive contact points with intersecting conductive warp threads and/or intersecting conductive weft threads of the moisture sensor.

A so-called carbon nanotube coating in which the reproducibility is especially reliable has been found to be an especially advantageous high-resistance polymer coating. Threads with such a carbon nanotube coating are described in the document EP 2 322 709 A1, for example.

According to the teaching of the invention, these problems are solved by a woven generic moisture sensor having at least one conductor produced by weaving which has an inventive monitoring section of the conductor which is produced by weaving. The monitoring section of the conductor consists of a special section of an electrically conductive thread.

A first embodiment of the present invention provides that the specific electrical resistance of the monitoring section of the conductor corresponds essentially to the specific electrical resistance of the threads of the conductor and that the monitoring section is separated from the conductor in a destructive manner after function testing of the conductor, so that the conductor is divided into at least two electrodes so that the moisture sensor becomes sensitive only through the step of separating the monitoring section for the moisture measurement. In this embodiment, a defect in the conductor, for example, an interruption due to a break in the conductor or a weaving defect can be detected when the dry moisture sensor is connected to the evaluation device, a known electrical test voltage is applied and the measured ohmic resistance between the contacts of the moisture sensor exceeds a predetermined first resistance limit value or falls below a second resistance cutoff value (short circuit) or when the electrical current is measured, the measured electrical current falls below a predetermined first current cutoff value (breakage of the conductor) or exceeds a second current cutoff value (short-circuit). The advantage of this first embodiment is that it is especially simple and inexpensive to produce the moisture sensor. However, then it is necessary to take into account the fact that a function test of the conductor is no longer possible after separating the monitoring section. However, tests have shown that the conductor cannot be damaged by the mechanical stresses on the moisture sensor to be expected in practical use on a patient.

A second embodiment of the present invention provides that the specific electrical resistance of the monitoring section of the conductor is must greater than the specific electrical resistivity of the threads of the conductor, and the monitoring section is embodied as a high-resistance terminating resistor. The specific electrical resistance of the monitoring section of the conductor is preferably between 10³ ohm/cm and 10⁶ ohm/cm. The specific electrical resistance of the monitoring section especially preferably amounts to 10⁴ ohm/cm and 10⁵ ohm/cm. The monitoring section of the conductor preferably consists of a thread with a polymer coating, in particular of a thread with a carbon nanotube coating. The high-resistance thread may pass through the entire woven sheeting due to the manufacturing process, for example, as a warp fiber in the direction of weaving or as a weft fiber across the direction of weaving. The length of the high-resistance thread which acts as a terminating resistor in the woven fabric is defined according to the invention by local conductive links with intersecting conductive weft fibers or intersecting conductive warped fibers. Therefore only a very precisely defined section of the high-resistance thread acts as a terminating resistor. The absolute amount of the effective high-resistance terminating resistor can be defined according to the invention only by weaving within predetermined tolerances.

### EXEMPLARY EMBODIMENTS OF THE PRESENT INVENTION

Exemplary embodiments of the inventive moisture sensor are explained in greater detail below with reference to the figures. Additional details and advantages of the invention are described in greater detail on the basis of exemplary embodiments depicted in the figures. The reference numerals in the figures all have the same meanings in the figures.

They show:

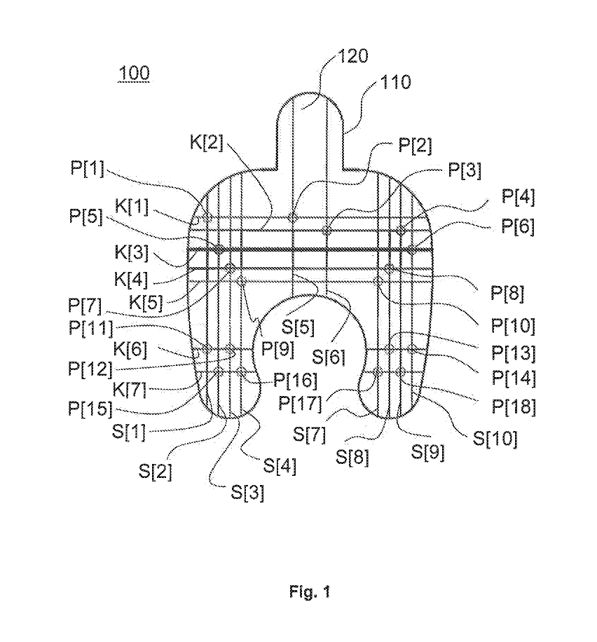

FIG. 1: schematic diagram of a woven moisture sensor according to a first exemplary embodiment

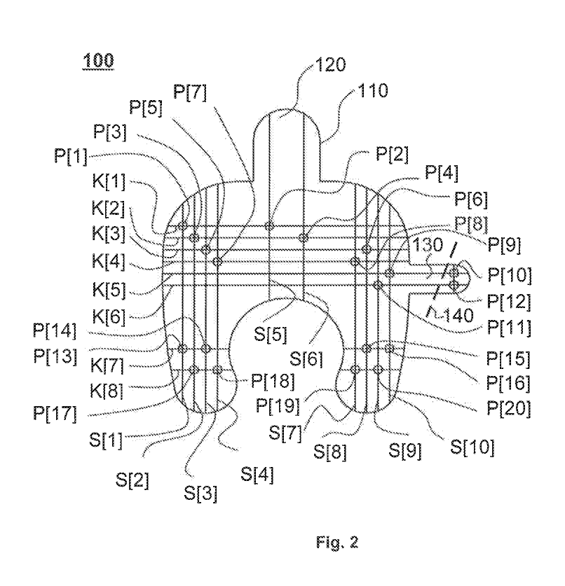

FIG. 2: schematic diagram of a woven moisture sensor according to a second exemplary embodiment

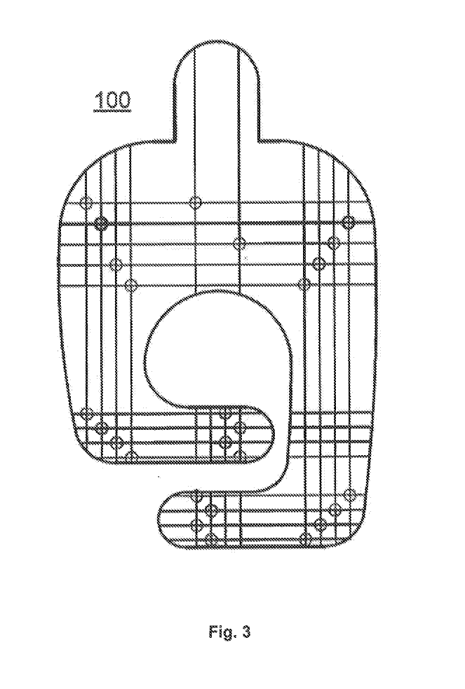

FIG. 3: schematic diagram of a woven moisture sensor according to a second exemplary embodiment

FIG. 1 shows in a simplified schematic diagram an inventive woven moisture sensor 100 which is bordered by the outer contour 110 and has a connecting tongue 120 for the electrical contacting of the moisture sensor with a terminal (not shown). The moisture sensor shown in the present exemplary embodiment is a refinement of the moisture sensor disclosed in the document WO 2011/116943 A1. In the woven moisture sensor 100, electrically conductive weft fibers S[1] through S[10] and electrically conductive warp fibers K[1] through K[7] are woven into a multilayer woven fabric. The electrically conductive warp fibers K[1] through K[7] are woven together with the electrically conductive weft fibers S[1] through S[10] so that they are electrically conductive only at the selected contact points P[1] through P[18] and are otherwise insulated from one another in the multilayer fabric. The weft fibers S[5] and S[6] run through the connecting tongue 120. Because of the contact points P[1] through P[18] this forms a closed conductor between the weft fibers S[5] in the connecting tongue 120 and the weft fiber S[6] in the connecting tongue 120. The course of the conductor is defined by the specific position of the contact points P[1] through P[18]. The closed conductor in the present exemplary embodiment of FIG. 1 consists of the fiber sections which are joined together in a conductive manner as listed below:
partial section in the weft fiber S[5] from the connecting tongue 120 to the contact point P[2],
from the contact point P[2] to the contact point P[1],
from the contact point P[1] to the contact point P[11],
from the contact point P[11] to the contact point P[12],
from the contact point P[12] to the contact point P[7],
from the contact point P[7] to the contact point P[8],
from the contact point P[8] to the contact point P[13],
from the contact point P[13] to the contact point P[14],
from the contact point P[14] to the contact point P[6],
from the contact point P[6] to the contact point P[5],
from the contact point P[5] to the contact point P[15],
from the contact point P[15] to the contact point P[16],
from the contact point P[16] to the contact point P[9],
from the contact point P[9] to the contact point P[10],
from the contact point P[10] to the contact point P[17],
from the contact point P[17] to the contact point P[18],
from the contact point P[18] to the contact point P[4],
from the contact point P[4] to the contact point P[3] and
from the contact point P[3] in the weft fiber S[6] as far as the connecting tongue 120.

In the present exemplary embodiment, the warp fiber K[3] is a high-resistance fiber (10⁵ ohm/cm in the exemplary embodiment), whereas all the other electrically conductive fibers have a low resistance. Due to the linkage of the warp fiber K[3] to the weft fiber S[2] at the contact point P[5] and the linkage of the warp fiber K[3] to the weft fiber S[10] at the contact point P[6], a high-resistance terminating resistor with a precisely defined length and with a resistance value specified within predetermined tolerances is created as a section of the conductor between the contact points P[5] and P[6]. The terminating resistor created in this way has a sufficient reproducibility in mass production and serves as the monitoring section for the function test of the moisture sensor in the dry state.

When the moisture sensor 100 is connected to an electrical evaluation unit (not shown) and an essentially constant test voltage is applied between the free ends of the weft fibers S[5] and S[6] on the connecting tongue, then an electrical current flows through the entire conductor.

In the dry state of the moisture sensor, the measured ohmic resistance of the conductor corresponds essentially to the previously known ohmic resistance of the terminating resistor. In the case of a break in the conductor or a defective or missing contacting of the connecting terminal (not shown) with the weft fibers in the connecting tongue, the measured ohmic resistance of the conductor is significantly higher than the previously known ohmic resistance of the terminating resistor, e.g., it is infinitely high. In the case of a moisture sensor, the measured ohmic resistance is much lower when moist than the previously known ohmic resistance of the terminating resistor. The measured ohmic resistance may be compared with a lower limit value and an upper limit value in the evaluation unit. If the measured ohmic resistance exceeds the upper limit value, then a defective sensor or a faulty connection of the moisture sensor to the evaluation unit is concluded. If the measured ohmic resistance falls below the lower limit value, then moisture is detected by the evaluation unit.

Similarly it is also possible to measure and evaluate the electrical current through the conductor. The measured electrical current may be compared with a lower limit value and an upper limit value in the evaluation unit. If the measured electrical current exceeds the upper limit value, then moisture is detected by the evaluation unit. If the measured electrical current falls below the lower limit value, then a defective sensor or a faulty connection of the moisture sensor to the evaluation unit is detected.

In connecting the dry moisture sensor to the evaluation unit, a basic measurement of the ohmic resistance and/or of the electrical current is performed for calibrating the evaluation unit and the lower limit values and the upper limit values are determined automatically with respect to the basic measurement. Empirical values from experiments with a large number of moisture sensors of the same type are stored in the evaluation unit for defining the lower limit values and the upper limit values with respect to the basic measurement.

The limited dimensions of a moisture sensor for monitoring a patient's vascular access restrict the available space for a woven terminating resistor. The shortest possible terminating resistor would be desirable for this reason. However, high-resistance threads in practice have deviations in the electrical properties, in particular the specific electrical resistance from the average manufacturer's specifications along the length of the threads. This means that good reproducibility of the electrical resistance value of a terminating resistor requires a certain minimum length through which the current effectively flows. Experience by the applicant has shown that there is a high reproducibility of the resistance values of a terminating resistor thereby produced when the length of the high-resistance thread through which the current is flowing is at least 10 mm but preferably approx. 50 mm or longer.

It is also possible to use an area of several threads as the terminating resistor instead of using a single thread as the terminating resistor.

In an alternative embodiment it is also possible to lengthen the segment of the terminating resistor through which the current flows through a design measure to improve the reproducibility.

In an alternative embodiment to the embodiment shown in FIG. 1, multiple high-resistance threads connected in parallel are woven into the fabric so that in this way the entire effective length of the high-resistance thread through which the current flows is increased.

In another alternative embodiment, an especially long section of the terminating resistor through which the electrical current flows can be achieved if a high-resistance thread is arranged along a portion of the outer contour of the moisture sensor and at two contact points on the outside contour with the ends of two low-resistance conductors which must not coincide with the low resistance conductors in the connecting tongue. However, such an arrangement would not be possible only through weaving in the case of a rounded outside contour, as shown in FIG. 1. However, in the case of a rectangular moisture sensor, this would be possible only through weaving. In such an arrangement of the terminating resistor, the intersection points of the high-resistance thread with the low-resistance threads are embodied as insulation points in the multilayer fabric.

In other embodiments additional design features are provided to increase the length of the section of the high-resistance thread through which the current flows. For example, a high-resistance company logo applied to it may be part of the woven high-resistance terminating resistor. However, this is possible by weaving alone only in certain embodiments.

FIG. 2 shows as a second exemplary embodiment a schematic diagram of a woven moisture sensor having an outer contour 110 and a connecting tongue 120 and a monitoring section 130 of the conductor produced by weaving for the function test of the conductor such that the monitoring section consists of a woven monitoring tongue 130 which protrudes essentially beyond the contour of the woven moisture sensor and contains a low-resistance conductor section which contains the contact points P[10] and P[12].

For the function test of the conductor, the resistance of the total low-resistance conductor can be measured. It is also possible to measure the electrical current through the low-resistance conductor in the function test and compare it with a lower limit value and an upper limit value. After conclusion of the function test the sensor is sensitized by cutting off the woven monitoring tongue 130 together with the contact points P[10] and P[12] from the woven moisture sensor along a line 140 indicated with a dotted line in FIG. 2, so that the closed conductor is separated into a first electrode and a second electrode. The first electrode and the second electrode are electrically insulated from one another after cutting off the monitoring tongue 130.

The first electrode extends over the interconnected thread sections:
partial section in the weft fiber S[5] from the connecting tongue 120 to the contact point P[2],
from the contact point P[2] to the contact point P[1],
from the contact point P[1] to the contact point P[13],
from the contact point P[13] to the contact point P[14],
from the contact point P[14] to the contact point P[5],
from the contact point P[5] to the contact point P[6],
from the contact point P[6] to the contact point P[15],
from the contact point P[15] to the contact point P[16],
from the contact point P[16] to the contact point P[9],
from the contact point P[9] in the warp thread K[5] to the cutting line 140.

The second electrode extends over the interconnected thread sections:
partial section in the weft thread S[6] from the connecting tongue 120 to the contact point P[4],
from the contact point P[4] to the contact point P[3],
from the contact point P[3] to the contact point P[17],
from the contact point P[17] to the contact point P[18],
from the contact point P[18] to the contact point P[7],
from the contact point P[7] to the contact point P[8],
from the contact point P[8] to the contact point P[19],
from the contact point P[19] to the contact point P[20],
from the contact point P[20] to the contact point P[11],
from the contact point P[11] in the warp thread K[6] to the cutting line 140.

The monitoring tongue 130 can be cut off with scissors, for example, after applying the moisture sensor to the patient's skin at a location close to the puncture site of the vascular access and checking it with regard to its function. The section of conductor between the contact points P[10] and P[12] was selected to be short to keep the loss of material minor and the effective electrode length long. To do so, the contact points P[10] and P[12] were formed with the warp threads K[5] and K[6], which are advantageously close to one another and one in parallel. Thus the monitoring tongue 130 may be designed to be small. Nevertheless the monitoring tongue is large enough to securely grip it while wearing sterile gloves when cutting it off on the patient and be able to cut if off with scissors.

In this embodiment, after separating the monitoring section from the moisture sensor, no further monitoring of the function is possible. However, stress tests performed by the applicant on woven moisture sensors have shown that woven moisture sensors, in particular those without high-resistance threads, are extremely resistant to the mechanical loads occurring in practical use in clinic. It is therefore sufficient to test the moisture sensor with regard to its function immediately prior to its use and then to avoid any other function tests during the extracorporeal blood treatment.

No high-resistance terminating resistor is needed in this embodiment. This is a cost advantage because no cost-intensive high-resistance threads are required.

FIG. 3 shows as the third exemplary embodiment a schematic diagram of a woven moisture sensor 100 resembling the first exemplary embodiment from FIG. 1, but with the additional feature that the two lateral legs of the moisture sensor are each designed with an elongation and each is guided at an angle around the central recess in the moisture sensor, wherein the angled regions of the two lateral legs are passed by one another at a distance and surround the central recess, so that the central recess in the moisture sensor is surrounded by sensitive regions on all sides without the angled regions of the two lateral legs coming in contact. The position of the contact points of the moisture sensor from FIG. 3 is adapted to the modified outer contour of the moisture sensor in comparison with FIG. 1. With the moisture sensor according to FIG. 3, it is possible to reduce the probability of liquid escaping from the region of the central recess between the two lateral legs without being detected.

According to the invention, the solution to the problems of the present invention succeeds with the exemplary embodiments presented here. However, the present invention is not limited to these exemplary embodiments.

### LIST OF REFERENCE NUMERALS

| Reference notation | Designation |
|---|---|
| moisture sensor | 100 |
| outside contour of the moisture sensor | 110 |
| connecting tongue | 120 |
| monitoring tongue | 130 |
| cutting line | 140 |
| weft threads | S[i], i = 1, . . . , n |
| warp threads | K[i], i = 1, . . . , m |
| contact points | P[i], i = 1, . . . , k |

1. A woven moisture sensor for monitoring a patient's vascular access having at least one conductor produced by weaving, having a monitoring section of the conductor which is produced by weaving for function-testing the conductor.

2. The woven moisture sensor according to claim 1, characterized in that the monitoring section of the conductor which is produced by weaving is configured as a terminating resistor.

3. The woven moisture sensor according claim 2, characterized in that the terminating resistor is a high-resistance electrically conductive thread and the specific electrical resistivity of the control thread is much greater than the specific electrical resistivity of the electrically conductive threads forming the conductor.

4. The woven moisture sensor according to claim 3, characterized in that the high-resistance electrically conductive threads have a specific electrical resistivity in the value range from 103 ohm/cm to 106 ohm/cm, in particular in the value range from 103 to 105 ohm/cm, based on the length of the thread.

5. The woven moisture sensor according to claim 4, characterized in that the high-resistance electrically conductive thread has a carbon nano tube coating.

6. The woven moisture sensor according to claim 1, characterized in that the monitoring section of the conductor which is produced by weaving is a section of the conductor which is provided and configured for being cut off from the conductor.

7. The woven moisture sensor according to claim 6, characterized in that the monitoring section of the conductor, which is configured and provided for separation from the conductor, is arranged in an area of the woven moisture sensor which is configured and provided for destructive separation.

8. The woven moisture sensor according to claim 7, characterized in that the area of the woven moisture sensor which is provided and configured for destructive separation is an area of the woven moisture sensor that is provided and configured for being cut off.

9. The woven moisture sensor according to claim 8, characterized in that: the area of the moisture sensor which is provided and configured for being cut off is a tongue.

10. An evaluation device for monitoring a patient's vascular access which is provided and configured for measuring the moisture with a moisture sensor according to claim 1 by measuring the electrical resistance between the terminal contacts of the moisture sensor and/or for measuring the electrical current in the conductor of the moisture sensor.

11. The evaluation device for monitoring a patient's vascular access according to claim 10, additionally configured for comparing the measured electrical resistance with a lower resistance limit value and/or an upper resistance limit value and/or for comparing the measured current with a lower current limit value and/or an upper current limit value.

12. The evaluation device for monitoring a patient's vascular access according to claim 11 with a moisture sensor having at least one conductor produced by weaving, with a monitoring section of the conductor produced by weaving for function-testing the conductor, characterized in that a defective conductor is inferred when the measured ohmic resistance falls below a first lower resistance limit value and/or the measured ohmic resistance exceeds a first upper resistance limit value and/or when the measured current falls below a first lower current limit, value and/or the measured current exceeds a first upper current limit value.

13. A blood treatment device having an extracorporeal blood circulation and an evaluation device according to claim 10.

14. A method for monitoring a patient's vascular access, having the steps:
applying a moisture sensor having at least one conductor produced by weaving, with a monitoring section of the conductor produced by weaving for function-testing the conductor close to the puncture site of the patient's vascular access,
contacting the moisture sensor with an evaluation unit which is provided and configured for measuring the moisture with said moisture sensor by measuring the electrical resistance,
measuring the electrical resistance of the moisture sensor and/or of the electrical current in the conductor,
monitoring the patient's vascular access by monitoring the measured electrical resistance of the moisture sensor and/or of the measured electrical current in the conductor.

15. The method for monitoring a patient's vascular access, having the steps:
applying a moisture sensor having at least one conductor produced by weaving, with a monitoring section of the conductor produced by weaving for function-testing the conductor close to the puncture site of the patient's vascular access,
contacting the moisture sensor with an evaluation unit which is provided and configured for measuring the moisture with said moisture sensor by measuring the electrical resistance,
measuring the electrical resistance of the moisture sensor and/or of the electrical, current in the conductor,
monitoring the patient's vascular access by monitoring the measured electrical resistance of the moisture sensor and/or of the measured electrical current in the conductor,
destructive separation, in particular cutting off the area of the woven moisture sensor which is provided and configured for destructive separation according to claim 7 before the start of the monitoring of the vascular access.
